(19) United States
(12) Patent Application Publication
(10) Pub. No.: US 2011/0262544 A1
(43) Pub. Date: Oct. 27, 2011

(54) OPHTHALMIC ADMINISTRATION OF A COMPOSITION INCLUDING BRIMONIDINE AS A MIST

(75) Inventors: Gilbert T. Feke, Stoncham, MA (US); Steven B. Koevary, Newton, MA (US); Yossi Gross, Moshav Mazor (IL)

(73) Assignee: PHARMALIGHT INC., Wilmington, DE (US)

(21) Appl. No.: 13/124,839

(22) PCT Filed: Oct. 21, 2009
(86) PCT No.: PCT/IB09/54656

§ 371 Date: Jun. 27, 2011

Related U.S. Application Data
(60) Provisional application No. 61/107,039, filed on Oct. 21, 2008.

(51) Int. Cl. A61K 9/14; A61B 19/00; A61P 27/06; A61K 31/498; A61P 27/02

(52) U.S. Cl. 424/489; 514/249; 206/438

(57) ABSTRACT

Disclosed are methods of treatment including administration of a pharmaceutical composition including brimonidine to an eye as a mist, the composition devoid of a penetration enhancer.

### RELATED APPLICATIONS

The present application gains priority from U.S. Provisional Patent Application No. 61/107,039 filed 21 Oct. 2008, which is included by reference as if fully set forth herein.

Some aspects of the present application are related to the teachings of PCT Patent Application No. PCT/IL2006/000145 filed 6 Feb. 2006 and published as WO 2006/082588 which claims the benefit of U.S. Provisional Patent Application No. 60/650,144 filed on 7 Feb. 2005; U.S. Provisional Patent Application No. 61/033,076 filed 3 Mar. 2008; and U.S. Provisional Patent Application No. 61/091,778 filed 26 Aug. 2008, all of the Applicant and all which are included by reference as if fully set forth herein.

### FIELD AND BACKGROUND OF THE INVENTION

The present invention, in some embodiments, relates to the field of ophthalmic medicine and more particularly to the ophthalmic administration of compositions including brimonidine as a mist.

The bulb of the eye (bulbus oculi; eyeball) is contained in the cavity of the orbit, where it is protected from injury. Associated with the eye are certain accessory structures such as the muscles, fascia, eyelids, conjunctiva, and lachrymal apparatus.

Only the surface of the anterior part of the eye, including the corneal epithelium and part of the episcleral conjunctiva, is exposed to the environment. The mucosa of the conjunctiva provides a protective interface between the eye and accessory structures. The exposed anterior surface is continuously washed by tear fluid. The nasolacrimal duct drains tears and other substances from the eye to be absorbed by a layer of mucosal membrane.

The eye is provided with blood through various retrobulbar arteries. The eye is extremely sensitive to any disruptions of its blood supply, which occur more frequently with age. Most disruptions of blood supply result at least partly from occlusion, for example due to atherosclerosis or an embolus, but may also occur as a result of inflammation of the blood vessels (vasculitis, such as temporal arteritis), inflammation of the optic nerve, infection in or around the eye, clotting disorders, damage from radiation, and injury to the eye. Disruption of blood flow to the eye generally results in vision loss, usually in one eye, which may be total or partial.

Reduced blood flow to the eye through the retrobulbar arteries has been associated with a number of ocular conditions, for example insufficient retrobulbar blood flow, diabetic retinopathy; glaucoma, ocular hypertension, macular degeneration, ocular ischemic syndrome, giant cell arteritis, eye occlusions, central retinal artery occlusion (CRAO), central retinal vein occlusion (CRVA), ischemic optic neuropathy, optic neuritis, neuromyelitis optica and neuroretinitis.

Glaucoma is a group of diseases of the optic nerve involving loss of retinal ganglion cells. The major risk factor for most types of glaucoma is an increased intraocular pressure (IOP): the higher the IOP the greater the likelihood of optic nerve damage.

The most common form of the disease, open-angle glaucoma, is distinguished by an increase in pressure inside the eye caused by gradual blockage of aqueous outflow due to clogging of the drainage system or over-production of aqueous fluid, and resulting in damage to the optic nerve and to the retina.

The link between glaucoma, intraocular pressure and abnormal retinal blood flow is not clear although it seems that persons suffering from glaucoma have insufficient autoregulation of retinal blood flow. It has been proposed that fluctuations in ocular perfusion pressure, and, thus, in blood flow, may lead to the damage to the retina and to the optic nerve observed in glaucoma patients.

Ocular hypertension is a condition in which the intraocular pressure is abnormally high, but no optic nerve damage is present and no signs of glaucoma are evident on visual field testing. Ocular hypertension may be due to, for example, traumatic hyphema, orbital edema, postoperative visco elastic retention, intraocular inflammation, corticosteroid use, pupillary block, or idiopathic causes.

Diabetic retinopathy is a complication of diabetes that results from damage to the retina. At first, diabetic retinopathy may cause no symptoms or only mild vision problems. Eventually, however, diabetic retinopathy can result in blindness. In the United States, diabetic retinopathy is a leading cause of blindness in adults.

Macular degeneration is a medical condition usually of older adults which results in a loss of vision in the center of the visual field (the macula) because of damage to the retina. It occurs in “dry” and “wet” forms. The “dry” form results from atrophy to the retinal pigment epithelial layer below the retina, which causes vision loss through loss of photoreceptors (rods and cones) in the central part of the eye. The “wet” form causes vision loss due to abnormal blood vessel growth in the choriocapillaries, through Bruch's membrane, ultimately leading to blood and protein leakage below the macula. Bleeding, leaking, and scarring from these blood vessels eventually causes irreversible damage to the photoreceptors and rapid vision loss if left untreated.

Ocular ischemic syndrome is caused by internal carotid artery atheromatous ulceration and stenosis at the bifurcation of the common carotid artery.

Giant cell arteritis is an inflammatory disease of blood vessels, often in the head.

When the inflammation affects the blood supply to the eyes, blurred vision or sudden blindness may occur.

Eye occlusions, also called eye strokes, occur when blood flow to important eye structures is blocked, for example, by a clot. For example, central retinal artery occlusion (CRAO) and central retinal vein occlusion (CRVA) occur when the artery or vein associated with the retina become occluded, potentially leading to complete loss of vision.

Ischemic optic neuropathy (both anterior and posterior ischemic optic neuropathy) is the loss of vision resulting from damage to a portion of the optic nerve due to obstruction of blood flow to the nerve (i.e., ischemia). In optic neuritis, inflammation of the optic nerve, especially of the myelin covering of the optic nerve, damages the nerve and may adversely affect vision. Optic neuritis is related to, associated with or may be caused by auto-immune diseases, such as multiple sclerosis, neuromyelitis optica, neuroretinitis, bacterial infections (e.g., Lyme's disease, cat scratch fever, syphilis), viral infections (e.g., HIV, hepatitis B, herpes), cranial arteritis, diabetes, drugs (such as ethambutol), radiation therapy, tumors, nutritional deficiencies, toxins and others.

A number of active pharmaceutical ingredients (API), topically administered to the eye in pharmaceutical compositions using eye-drops, are known for treating ocular conditions by reducing intraocular pressure and/or improving ocular blood flow.

For example, compositions including the alpha2-adrenergic agonist brimonidine or salts thereof as an API are used to decrease production of aqueous humor as well as to increase uveoscleral outflow, to reduce intraocular pressure.

Glaucoma patients maintained retinal blood flow homeostasis with changes in posture when treated with brimonidine but not with latanoprost (Pasquale L R, Feke G T, Menke M N, Kuperwaser M, McMeel J W in “Effect of Brimonidine versus Latanoprost on the Maintenance of Retinal Blood Flow Homeostasis during Postural Change in Normal Tension Glaucoma” Investig Ophthalmol Vis Sci 2004, 45, E-Abstract 1178 and presented at the 2004 meeting of the Association for Research in Vision and Ophthalmology.

Thus, it seems that alpha2-adrenergic agonists, such as brimonidine, may be exceptionally useful for treating glaucoma, both by directly reducing intraocular pressure and by restoring autoregulation of retinal blood flow.

Alphagan® P by Allergan Inc. (Irvine, Calif., USA) is a commercially-available composition administered by instillation as eye-drops prescribed for treating open-angle glaucoma or ocular hypertension by ophthalmic administration, which includes brimonidine tartrate (5-bromo-6-(2-imidazolidinylideneamino) quinoxaline L-tartrate) as an API.

Poor patient compliance with the administration regimen is considered as one of the main reasons for failure of treatment of ocular conditions by the use of eye drops, including with brimonidine. This is attributed to both the general inconvenience of using eye drops as well as to the ocular irritation caused using existent topical pharmaceutical compositions prescribed for glaucoma and ocular hypertension, such as Brimonidine-containing compositions.

In PCT patent publication WO 2006/082588 of the Applicant is disclosed that the ocular irritation caused by a pharmaceutical composition including a penetration enhancer is reduced when the composition is administered to the eye as a mist. Specifically, administration of a brimonidine-containing composition including a highly irritant penetration enhancer (e.g., saponin, fusidate, azone, bile acid salts such as glycholate and cholate) as a mist is disclosed.

### SUMMARY OF THE INVENTION

Aspects of the invention relate to pharmaceutical compositions for ophthalmic administration as a mist, the compositions including brimonidine as an active pharmaceutical ingredient and substantially devoid of penetration enhancers. As used herein, by “brimonidine” is intended brimonidine (5-Bromo-N-(4,5-dihydro-1H-imidazol-2-yl) quinoxalin-6-amine), pharmaceutically acceptable salts thereof (e.g., brimonidine tartrate) and combinations thereof.

According to an aspect of some embodiments of the invention there is provided a method for the treatment of a condition of the eye, comprising: administering an effective amount of a pharmaceutical composition comprising brimonidine or a pharmaceutically acceptable salt thereof in an ophthalmically-acceptable carrier to the eye of a subject as a mist, the composition substantially devoid of a penetration enhancer thereby treating the condition.

According to an aspect of some embodiments of the invention there is also provided a method of treatment of a condition of the eye, comprising: a) providing a pharmaceutical composition comprising brimonidine or a pharmaceutically acceptable salt thereof, and an ophthalmically acceptable carrier, the composition substantially devoid of a penetration enhancer; b) generating a mist of the composition; and c) contacting the mist with an anterior surface of the eye of a subject.

According to an aspect of some embodiments of the invention there is also provided the use of a mist for ophthalmic delivery of a pharmaceutical composition comprising brimonidine or a pharmaceutically acceptable salt thereof, and an ophthalmically acceptable carrier to a subject in need of treatment of a condition, the composition substantially devoid of a penetration enhancer.

According to an aspect of some embodiments of the invention there is also provided the use of a pharmaceutical composition comprising brimonidine or a pharmaceutically acceptable salt thereof and an ophthalmically acceptable carrier in the form of a mist for the treatment of a condition of the eye of a subject, wherein the composition is substantially devoid of a penetration enhancer.

According to an aspect of some embodiments of the invention there is also provided the use of brimonidine or a pharmaceutically acceptable salt thereof in the preparation of a pharmaceutical composition for treatment of a condition by administration of the pharmaceutical composition as a mist to the eye of a subject.

In some embodiments, the condition is selected from the group consisting of a condition susceptible to stimulation of retrobulbar blood flow and a condition susceptible to lowering of intraocular blood pressure.

In some embodiments, the condition is a condition susceptible to stimulation of retrobulbar blood flow selected from the group consisting of diabetic retinopathy; glaucoma, ocular hypertension, macular degeneration, ocular ischemic syndrome, giant cell arteritis, eye occlusions, central retinal artery occlusion (CRAO), central retinal vein occlusion (CRVA), ischemic optic neuropathy, optic neuritis, neuromyelitis optica and neuroretinitis. In some embodiments, the condition susceptible to lowering of intraocular blood pressure is selected from the group consisting of glaucoma and ocular hypertension, such as open angle glaucoma.

In some embodiments, the subject is a human. In some embodiments, the subject is a non-human animal, e.g., a horse, a cat, a dog, a cow, a sheep or a pig.

According to an aspect of some embodiments of the invention there is also provided a device for ophthalmic administration of a pharmaceutical composition, comprising: a) a composition reservoir configured for functional association with a nebulizing device; and b) a pharmaceutical composition comprising brimonidine or a pharmaceutically acceptable salt thereof and an ophthalmically acceptable carrier contained in the reservoir, wherein the composition is substantially devoid of a penetration enhancer, wherein the nebulizing device includes a nebulizer configured to nebulize the pharmaceutical composition so as to generate an ophthalmically administrable mist.

According to an aspect of some embodiments of the invention there is also provided a pharmaceutical composition, comprising: a) brimonidine or a pharmaceutically acceptable salt thereof; and b) an ophthalmically-acceptable carrier, the composition configured for ophthalmic administration as a mist; and the composition substantially devoid of a penetration enhancer.

In some embodiments, the mist comprises particles having a mean particle diameter of less than about 20 micrometers, less than about 10 micrometers, less than about 8 micrometers, less than about 5 micrometers, less than about 3 micrometers and even less than about 1 micrometer.

In some embodiments, the pharmaceutical composition further comprises one or more additional components, for example a buffering agents, pH-adjusting agents, preservatives, and solubilizers.

In some embodiments where the composition includes a buffering agent, the buffering agent is selected from the group consisting of borate buffers, citrate buffers, acetic acid/sodium acetate buffers, phosphoric acid/sodium phosphate buffers, mannitol, or combinations thereof.

In some embodiments where the composition includes a pH-adjusting agent, the pH-adjusting agent is selected from the group consisting of adipic acid, boric acid, citric acid, glycine, calcium hydroxide, magnesium aluminometasilicates, hydrochloric acid, lactic acid, phosphoric acid, sodium hydroxide, sorbic acid, sulfuric acid and tartaric acid, derivatives thereof, salts thereof, or combinations thereof.

In some embodiments where the composition includes a preservative, the preservative is selected from the group consisting of propylene glycols, sodium propionate, sodium perborate, chlorine dioxide, vitamin E, vitamin E acetate and derivatives, esters, salts, or combinations thereof.

In some embodiments where the composition includes a solubilizer, the solubilizer is selected from the group consisting of citric acid, ethylenediamine-tetraacetate, sodium meta-phosphate, succinic acid, urea, cyclodextrin, polyvinylpyrrolidone, diethylammonium-ortho-benzoate, micelle-forming solubilizers, TWEEN, SPANS, polyoxyethylene sorbitan fatty acid ester, polyoxyethylene n-alkyl ethers, n-alkyl amine n-oxides, poloxamers, phospholipids and cyclodextrins, or combinations thereof.

In some embodiments, the composition further comprises a bioadhesive or viscosity modifier, for example selected from the group consisting of polyvinyl alcohol, thiolated poly acrylic acid, carbomer and gellan gum, methylcellulose and polyvinylpyrrolidone or combinations thereof.

Unless otherwise defined, all technical and scientific terms used herein have the same meaning as commonly understood by one of ordinary skill in the art to which this invention pertains. In case of conflict, the patent specification, including definitions, will control.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

### DESCRIPTION OF SOME EMBODIMENTS OF THE INVENTION

Aspects of the invention relate to methods of treating a condition of the eye by administering a pharmaceutical composition including brimonidine substantially devoid of highly-irritant penetration enhancers and even substantially devoid of any penetration enhancer as a mist to an eye of a subject in need thereof.

Aspects of the invention relate to use of a pharmaceutical composition in the form of a mist, for the treatment of a condition of the eye, the composition including brimonidine and being substantially devoid of highly-irritant penetration enhancers and even substantially devoid of any penetration enhancer.

Aspects of the invention relate to the administration to the eye of a pharmaceutical composition as a mist comprising brimonidine substantially devoid of highly-irritant penetration enhancers and even substantially devoid of any penetration enhancer in order to achieve a beneficial effect.

Aspects of the invention relate to pharmaceutical compositions including brimonidine substantially devoid of highly-irritant penetration enhancers and even substantially devoid of any penetration enhancer, for administration as a mist.

Aspects of the invention relate to methods and devices relating to the administration of a pharmaceutical composition comprising brimonidine in an ophthalmically- acceptable carrier as a mist, the composition substantially devoid of highly-irritant penetration enhancers and even substantially devoid of any penetration enhancer to an eye of a subject in need thereof.

As used herein a “pharmaceutical composition” refers to a preparation of brimonidine and/or pharmaceutically acceptable salts thereof as an active pharmaceutical ingredient, with other components such as pharmaceutically suitable carriers and excipients.

As used herein, the term “pharmaceutically acceptable” and “physiologically acceptable” are equivalent and mean approved by a regulatory agency of the Federal or a state government or listed in the U.S. Pharmacopeia or other generally recognized pharmacopeia for use in animals, and more particularly in humans. Herein, the phrases “physiologically suitable carrier” and “pharmaceutically acceptable carrier” are interchangeably used and refer to an approved carrier or a diluent that does not cause significant irritation to an organism and does not abrogate the pharmaceutical activity and properties of the administered active pharmaceutical ingredient.

As used herein, the term “carrier” refers to a diluent, adjuvant, excipient, or vehicle with which the active pharmaceutical ingredient is administered.

As used herein, the term “substantially devoid of a highly-irritant penetration enhancer” means that the composition comprises less than 0.05%, less than about 0.03% and even less than about 0.02% by weight of a highly-irritant penetration enhancer.

As used herein, the term “substantially devoid of a penetration enhancer” means that the composition comprises less than about 0.05%, less than about 0.03% and even less than about 0.02% by weight of a penetration enhancer.

At such low concentrations, the amount of penetration enhancer in the compositions is less than the minimal amount required to substantially increase the amount or rate of absorption into the body of a substance coadministered therewith.

As used herein, the term “method” refers to manners, means, techniques and procedures for accomplishing a given task including, but not limited to, those manners, means, techniques and procedures either known to, or readily developed from known manners, means, techniques and procedures by practitioners of the relevant arts. Implementation of the methods of the present invention involves performing or completing selected tasks or steps manually, automatically, or a combination thereof.

As used herein, the phrase “treating a condition” includes curing a condition, preventing a condition, treating symptoms of a condition, curing symptoms of a condition, ameliorating symptoms of a condition, treating effects of a condition, ameliorating effects of a condition, and preventing results of a condition.

Generally, the conditions of the eye treated are conditions susceptible to treatment with brimonidine or salts thereof, for example, conditions susceptible to stimulation of retrobulbar blood flow and/or conditions susceptible to lowering of intraocular blood pressure.

Conditions susceptible to stimulation of retrobulbar blood flow include, for example, diabetic retinopathy; glaucoma, ocular hypertension, macular degeneration, ocular ischemic syndrome, giant cell arteritis, eye occlusions, central retinal artery occlusion (CRAO), central retinal vein occlusion (CRVA), ischemic optic neuropathy, optic neuritis, neuromyelitis optica and neuroretinitis.

Conditions susceptible to lowering of intraocular blood pressure include glaucoma (such as open angle glaucoma) and ocular hypertension.

The principles, uses and implementations of the teachings of the invention may be better understood with reference to the accompanying description. Upon perusal of the description herein, one skilled in the art is able to implement the teachings of the invention without undue effort or experimentation.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

Brimonidine is an API effective for treating open-angle glaucoma or ocular hypertension. Known pharmaceutical compositions of brimonidine in the form of eye drops (e.g., Alphagan® P from Allergan Inc., Irvine, Calif., USA) are “ophthalmically gentle”, including brimonidine tartrate (0.1% or 0.15%), sodium carboxymethylcellulose (viscosity enhancer), sodium borate, boric acid, sodium chloride, potassium chloride, calcium chloride, magnesium chloride and 0.05 mg/ml Purite® (sodium perborate) as a preservative in an aqueous solution with HCl and/or NaOH added to adjust the pH to 7.4-8.0 (0.1%) or 6.6-7.4 (0.15%).

Despite the “gentle” composition, adverse reactions are surprisingly common: up to 20% of subjects suffer from allergic conjunctivitis, conjunctive hyperemia and eye pruritus. Other undesirable side-effects include itching, burning and stinging of the eye, redness of the eye or inner lining of the eyelid, swelling of the eyelid, tearing of the eye and less commonly ache or pain in eye, bloody eye, drainage from the eye, oozing in eye and swelling of eye (see insert supplied with Alphagan® P). Such side-effects lead to low patient compliance and failure of treatment.

Furthermore, although technically simple, instillation of eye drops has many disadvantages which adversely affect patient compliance. Receiving eye drops requires practice: it is unpleasant to open an eye widely while the drop is instilled, for adults but especially for children. Self-administration is not simple and often not effective when a drop is inaccurately placed. Often a person will instill more than the required number of drops, whether by accident or intent, and drops have a notoriously poorly defined volume making accurate dosage virtually impossible (Lederer, C. M. Jr. et al. in American Journal of Ophthalmology 1986, 101(6), 691-694 reports between 25 and 56 μl). Inadvertent contact of an eye dropper with the eye frequently occurs, potentially damaging the eye and compromising sterility.

In PCT patent publication WO 2006/082588 of the Applicant is disclosed administration of a brimonidine composition including a highly irritant penetration enhancer (e.g., saponin, fusidate, azone, bile acid salts such as glycholate and cholate) as a mist. The coadministration of brimonidine with a penetration enhancer as a mist was considered to be necessary for increasing the bioavailability of the brimonidine, allowing more effective treatment of conditions such as glaucoma and ocular hypertension, and allows administration of a reduced dose of API.

Aspects of the invention are based on the unexpected discovery that administration of a pharmaceutical composition comprising brimonidine to the eye as a mist where the composition is substantially devoid of highly-irritant penetration enhancers and even substantially devoid of any penetration enhancer achieves a beneficial effect and has advantages over prior art administration of brimonidine compositions, for example as disclosed in PCT patent publication WO 2006/082588 of the Applicant.

The fact that a composition for implementing the invention is substantially devoid of an additional ingredient makes manufacture of the composition simpler and cheaper. There is a reduced risk of adverse reaction by subjects who have a specific sensitivity or allergy to the additional ingredient.

Formulation of a pharmaceutical composition suitable for ophthalmic delivery as a mist is within the ability of a person having ordinary skill in the art using techniques with which one of average skill is familiar, which are discussed in numerous reference works, such as Remington's Pharmaceutical Science 15^{th} Edition.

Administration of a given embodiment of a pharmaceutical composition of the invention is generally performed by generating a mist from the composition and contacting the mist with an anterior surface of the eye. Herein, the term “mist” refers to a cloud of particles having a mean particle diameter of less than about 20 micrometers, less than about 10 micrometers, less than about 8 micrometers, less than about 5 micrometers, less than about 3 micrometers and even less than about 1 micrometer.

All things being equal, preliminary results indicate that finer mists provide superior results than do denser mists.

Embodiments of the invention are implemented where the mist is directed preferentially or selectively towards exposed portions of the sclera.

Mists are generated, for example, with a nebulizer. Herein, the term “nebulizer” is understood to mean a device or a part of a device that converts a substance, e.g., a solid, gel, liquid, solution, suspension, ointment, pharmaceutical composition, into a mist.

A pharmaceutical composition of the invention may be administered as a mist to an eye using any suitable nebulizing device known in the art for ophthalmic administration of pharmaceutical compositions. Suitable such nebulizing devices (as well as guidance in preparing compositions suitable for nebulizing) are well-known in the art and include embodiments of nebulizing devices described in the PCT patent publication WO2006/082588 of the Applicant, in U.S. Pat. No. 6,748,977, in US Patent Application 2007/0119968, in Collins J F et al in American Journal of Ophthamology 2007, 144(1), 137-139 or the ophthalmic delivery device by Optimyst, Llc (West Islip, N.Y., USA).

Suitable nebulizing devices generally include a nebulizer functionally associated with a composition-reservoir containing the pharmaceutical composition. When the nebulizing device is activated, composition is drawn from the reservoir and nebulized by the nebulizer to generate a mist. Once generated, the mist may then be administered to an eye to implement some embodiments of the methods and uses described herein.

A nebulizing device suitable for implementing the teachings herein may optionally comprise features useful for an ophthalmic delivery system including dosage control, employ a computerized system to program a predetermined medication-application regimen, call or beep the user, at the scheduled time for medication application, store data related to the medication application, and communicate with a computer or a health clinic concerning the treatment regimen for follow-up and evaluation. A suitable nebulizing device may optionally include a feature to direct a generated mist only at an open eye, increasing dosage accuracy and reducing composition wastage. In some embodiments a suitable nebulizing device may optionally include a self-sterilizing feature increasing the safety of the device and allowing the device to be easily used in hospitals and in situations that require high-throughput administration of a pharmaceutical composition.

According to an aspect of some embodiments of the invention, there is provided a device comprising a pharmaceutical composition of the invention contained in a composition reservoir, the composition reservoir configured for functional association with a nebulizing device including a nebulizer, the device suitable for ophthalmic administration of a composition as a mist. In some embodiments, the composition-reservoir containing the pharmaceutical composition is packaged in a packaging material or is labeled and identified in print, in or on the packaging material, as an ophthalmically deliverable composition as a mist for use for a need, as described above.

In such embodiments, the nebulizer is configured to nebulize the pharmaceutical composition so as to generate an ophthalmically administrable mist. In some embodiments, the composition reservoir holds a single dose of the composition. In some embodiments, the composition reservoir holds more than one dose of the composition.

In some such embodiments, the pharmaceutical composition is provided in a separate container or package and transferred into the composition reservoir of a suitable nebulizing device, for example by pouring. Once the pharmaceutical composition is contained inside the composition reservoir and the device is activated, composition is drawn from the reservoir and nebulized by the nebulizer to generate a mist for delivery to an eye.

In some such embodiments, the composition reservoir containing the pharmaceutical composition is provided separately from a nebulizer, for example, in some embodiments, the reservoir is provided packaged separately from the nebulizer, for example as a cartridge or a disposable cartridge. For use, the composition reservoir containing the pharmaceutical composition is removed from any extraneous packaging (e.g., a box or sealed envelope) and functionally associated with a suitable nebulizing device, for example by screwing or pushing into an appropriately configured port of the nebulizing device. In some embodiments, a seal or cover of the container is opened prior to the functional association. In some embodiments, the act of functional association with the nebulizing device breaks a seal or opens the container. Once the composition reservoir is functionally associated with the nebulizing device, when the nebulizer of the nebulizing device is activated, composition is drawn from the reservoir and nebulized to generate a mist. Once generated, the mist may then be administered to an eye to implement embodiments of the methods and uses described herein.

In some such embodiments, the composition reservoir containing the pharmaceutical composition and the nebulizing device are provided together, in some embodiments in the same package, in some embodiments where the composition reservoir is substantially fixed to the nebulizing device analogous to inhalers used for administering medication to asthma sufferers. In some such embodiments, a user opens a package containing a single unit that is substantially ready for use, comprising the nebulizer device and a functionally associated composition-reservoir containing the pharmaceutical composition.

Embodiments of the invention relate to methods of treatment, comprising administering to the eye as a mist an effective amount of a pharmaceutical composition comprising brimonidine in an ophthalmically- acceptable carrier, the composition substantially devoid of highly-irritant penetration enhancers and even substantially devoid of any penetration enhancer. The amount of composition administered as a mist and the length of time of administration is dependent on many factors, including the concentration of active ingredient, the nature and severity of the condition treated, the amount of composition wasted (deposited on the eyelid and the like) which is dependent on the nature of the nebulizing device. That said, the amount of composition nebulized in a single administration session is generally between about 10 and about 200 microliters, administered over a period of between about 15 and about 200 seconds.

Embodiments of the invention relate to the use of a pharmaceutical composition comprising brimonidine and an ophthalmically acceptable carrier in the form of a mist for the treatment of a condition of the eye selected from the group consisting of a condition susceptible to stimulation of retrobulbar blood flow and a condition susceptible to lowering of intraocular blood pressure, wherein the composition is substantially devoid of highly-irritant penetration enhancers and even substantially devoid of any penetration enhancer.

Embodiments of a composition of the invention include brimonidine in an ophthalmically acceptable carrier and optionally other ingredients, but substantially devoid of highly-irritant penetration enhancers and even substantially devoid of any penetration enhancer.

Generally, the concentration of the active ingredient (brimonidine or salt) is any suitable concentration. In some embodiments, the concentration is similar to that of Alphagan® P, that is to say between about 0.01% and about 0.3%, and in some embodiments, between about 0.05% and about 0.2%. That said, in some embodiments, the concentration of active ingredient is higher, in some embodiments at least about 0.3%, at least about 0.5% and even at least about 0.7%, allowing administration of smaller volumes of composition and/or achieving greater efficacy. Pharmaceutical compositions having such high concentrations of brimonidine are impractical for administration using eye drops due to the irritation caused to the eye by the brimonidine. However, apparently the mist delivery of the pharmaceutical composition in accordance with the teachings herein avoids or reduces the extent of ocular irritation caused by the brimonidine.

As used herein, the term “ophthalmically acceptable carrier” describes a carrier that does not cause significant irritation to the eye of an organism when applied in accordance with the teachings of the present invention and does not abrogate the pharmacological activity and properties of an API carried therewith.

Ophthalmically acceptable carriers are generally sterile, essentially free of foreign particles, and generally have a pH in the range of 5-8. Preferably, the pH is as close to the pH of tear fluid (7.4) as possible. Ophthalmically acceptable carriers are, for example, sterile isotonic solutions such as isotonic sodium chloride or boric acid solutions. Such carriers are typically aqueous solutions contain sodium chloride or boric acid. Also useful are phosphate buffered saline (PBS) solutions. Exceptionally suitable is the ophthalmically acceptable carrier used in formulating Alphagan® P, as described above.

In some embodiments, the composition administered as a mist in accordance with the teachings of the invention is Alphagan® P.

In some embodiments, the composition comprises a modification of Alphagan® P, being devoid of a viscosity modifier or bioadhesive. An exemplary composition according to this embodiment comprises brimonidine tartrate, sodium borate, boric acid, sodium chloride, potassium chloride, calcium chloride, magnesium chloride and sodium perborate.

In embodiments of the invention, a composition includes an effective amount of brimonidine or a pharmaceutically acceptable salt thereof.

In some embodiments, the pharmaceutically acceptable salt of brimonidine comprises brimonidine tartrate.

Preparation of a pharmaceutical composition of the invention is performed in the usual way, generally comprising mixing the various components together in the proper proportions. Thus, according to an aspect of some embodiments of the invention, there is provided the use of brimonidine or a pharmaceutically acceptable salt thereof in the preparation of a pharmaceutical composition for treatment of condition by ophthalmic administration of the pharmaceutical composition as a mist.

An effective amount of brimonidine or a salt thereof, as used herein, means an amount needed to achieve the desired need as described above, for example, a desired prophylactic, therapeutic or pharmaceutical effect. Determination of the effective amount, and consequently the dose and dose frequency, is within the capability of one skilled in the art, in light of the disclosure provided herein. Generally, medical personnel such as a doctor prescribing a pharmaceutical composition for use in accordance with the teachings of the invention prescribe a dosage regime including one or more administrations of a dose of the composition over a period of time (e.g., once a day, twice a day, three times a day). The dosage regime is generally chosen to be effective, that is to say sufficient to achieve a desired beneficial effect, e.g., to treat a condition.

Determination of an effective dosage regime is within the capability of a person having ordinary skill in the art in light of the disclosure provided herein for example using techniques with which one of average skill is familiar, which are discussed in numerous reference works, such as Remington's Pharmaceutical Science 15th Edition. Factors in determining the dosage regime vary with the type of the condition as well as such factors as the concentration of the ocular irritant, the subject being treated, the severity of the condition, the age, body weight and response of an individual patient and the judgment of the prescribing physician.

In some embodiments, administration is of lesser, equal or greater amounts of brimonidine or a salt thereof, than known in the art with fewer adverse reactions.

In some embodiments, administration is of lesser, equal or greater amounts of brimonidine or a salt thereof, than known in the art with increased efficacy.

In some embodiments, administration for a sufficient beneficial effect is less than the three-times daily required by the art.

Embodiments of compositions of the invention are substantially devoid of highly-irritant penetration enhancers and even substantially devoid of any penetration enhancer. Penetration enhancers are materials that increase the amount or rate of absorption into the body of a substance coadministered therewith. Penetration enhancers are materials that transiently increase the permeability of the corneal epithelium or conjunctiva to facilitate API penetration therethrough. The use of known percutaneous penetration enhancers in pharmaceutical compositions for ophthalmic administration has been proposed (see Sasaki et al.Crit. Rev. Ther. Drug Carrier Syst. 1999, 16, 85-146 and PCT patent publication WO 2006/082588).

Penetration enhancers can be classified as being:

a) inherently highly irritating to the eye; or

b) mildly irritating to the eye but highly irritating at high concentrations.

As used herein, highly irritating penetration enhancers are saponin and saponin derivatives, benzalkonium chloride, BL-9, deoxycholic acid, digitonin, escin, fusidic acid, fusidate, fusidic acid derivatives, sodium deoxycholate, acetone, acyl lactylates, acyl peptides, acylsarcosinates, alcohols, alkanolamine salts of fatty acids, alkyl benzene sulphonates, alkyl ether sulphates, alkyl sulphates, allantoin, anionic surface-active agents, 1-substituted azacycloheptan-2-ones, benzyl benzoate, benzyl salicylate, butan-1,4-diol, butyl benzoate, butyl laurate, butyl myristate, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl) ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dioctyl adipate, dioctyl azelate, dioctyl sebacate, 1,4 dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, glycerol monolaurate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, lecithin, maypons, metal salts of fatty acids, methyl nicotinate, 2-methyl propan-2-ol, 1-methyl-2-pyrrolidone, 5-methyl-2-pyrrolidone, methyl taurides,miranol, nonionic surface-active agents, octyl alcohol, octylphenoxy polyethoxyethanol, oleic ethanolamide, pleyl alcohol, pentan-2,4-diol, pheno xyethanol, phosphatidyl choline, phosphine oxides, polyalkoxylated ether glycollates, poly(diallylpiperidinium chloride), poly(dipropyldiallylammonium chloride), polyethylene glycol monolaurate, polyglycerol esters, poly(vinyl pyridinium chloride), propan-1-ol, propan-2-ol, propylene glycol, propylene glycol dipelargonate, propylene glycol monolaurate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quaternised poly (dimethylaminoethylmethacrylate), quaternised poly (vinyl alcohol), sapamin hydrochloride, sodium cocaminopropionate, sodium dioctyl sulphonsuccinate, sodium laurate, sodium lauryl ether sulphate, sodium lauryl sulphate, sorbitan monooleate, sorbitan monolaurate, sugar esters, sulpho succinate, tetrahydro furan, tetrahydrofurfural alcohol, transcutol, triethanolamine dodecyl benzene sulphonate, triethanolamine oleate, urazole, urea and derivatives, esters, salts and mixtures thereof.

As used herein, mildly irritating penetration enhancers that are highly irritating at high concentrations are ammonium glycyrrhizide, Brij 35, Brij 78, Brij-98, cetylpyridium chloride, chenodeoxycholic acid, cholate, cholic acid, decamethonium, decamethonium bromide, dimethyl sulphoxide, EDTA and disodium EDTA, glycocholate, glycocholic acid, glycodeoxycholic acid, glycyrrhizic acid, paraben, polyoxyethylene, polyoxyethylene ethers of fatty acids such as polyoxyethylene 4-, 9-, 10-, and 23-lauryl ether, polyoxyethylene 10-and 20-cetyl ether, polyoxyethylene 10- and 20-stearyl ether, polyoxyethylated castor oil, polyoxyethylene monolaurate, polyoxyethylene sorbitans such as polyoxyethylene sorbitan monolaurate, polyoxy:polyoxyethylene stearate, polyoxypropylene 15 stearyl ether, sodium cholate, sodium glycocholate, sodium taurocholate, sodium glyco deo xycho late, sodium tauro deo xycho late, sodium urso deo xycho late, taurocholic acid, tauro deo xycho lic acid, TWEEN 20, urosdeoxycholic acid, and derivatives, esters, salts and mixtures thereof.

It is often desired to provide a pharmaceutical composition with additional useful properties. Therefore, in embodiments, a composition of the invention includes, in addition to brimonidine, at least one additional component. It is important to note that in some cases a specific additional component also serves as a component of the carrier or serves two or more additional functions. Typical additional components include but are not limited to buffering agents, pH-adjusting agents, preservatives, solubilizers and viscosity modifiers.

In embodiments of the invention, a composition includes a buffering agent. Suitable buffering agents include but are not limited to borate buffers, citrate buffers, acetic acid/sodium acetate buffers and a phosphoric acid/sodium phosphate buffers. Embodiments of the composition that include a buffering agent that is an irritating penetration enhancer as listed above, include that buffering agent in an amount less than 0.05% by weight of the composition.

In embodiments of the invention, a composition includes a pH-adjusting agent. Suitable pH-adjusting agents include but are not limited to adipic acid, boric acid, citric acid, glycine, calcium hydroxide, magnesium aluminometasilicates, hydrochloric acid, lactic acid, phosphoric acid, sodium hydroxide, sorbic acid, sulfuric acid and tartaric acid, derivatives thereof, salts thereof or combinations thereof. Embodiments of the composition that include a pH-adjusting agent that is an irritating penetration enhancer as listed above, include that pH-adjusting agent in an amount less than 0.05% by weight of the composition.

In embodiments of the invention, a composition includes an ophthalmically acceptable preservative such as propylene glycols, sodium propionate, sodium perborate, chlorine dioxide, vitamin E, vitamin E acetate and derivatives, esters, salts, or combinations thereof. Embodiments of the composition that include a preservative that is an irritating penetration enhancer as listed above, include that preservative in an amount less than 0.05% by weight of the composition.

In embodiments of the invention, a composition includes a solubilizer. Examples of suitable solubilizers include citric acid, ethylenediamine-tetraacetate, sodium meta-phosphate, succinic acid, urea, cyclodextrin, polyvinylpyrrolidone, diethylammonium-ortho-benzoate, micelle-forming solubilizers, SPANS, polyoxyethylene sorbitan fatty acid ester, polyoxyethylene n-alkyl ethers, n-alkyl amine n-oxides, poloxamers, phospholipids and cyclodextrins, or combinations thereof. Embodiments of the composition that include a solubilizer that is an irritating penetration enhancer as listed above, include that solubilizer in an amount less than 0.05% by weight of the composition.

In embodiments of the invention, a composition further includes a bioadhesive or viscosity modifier, that is useful to keep an administered API a longer than usual time on the cornea, or a viscosity modifier. Suitable bioadhesives or viscosity modifiers include but are not limited to polyvinyl alcohol, thiolated poly acrylic acid, carbomer and gellan gum, methylcellulose and polyvinylpyrrolidone. Embodiments of the composition that include a bioadhesive that is an irritating penetration enhancer as listed hereinabove, include that bioadhesive in an amount less than 0.05% by weight of the composition.

It is preferred that a pharmaceutical composition of the invention be packaged in a packaging material and identified in print, in or on the packaging material, as a pharmaceutical composition ophthalmically deliverable as a mist for use for a need.

### EXAMPLES

Reference is now made to the following examples, which together with the above descriptions illustrate some embodiments of the invention in a non limiting fashion.

### Example 1

### Effect of Compositions of the Invention on Retinal Blood Flow

Retinal blood flow rate in the major temporal vein in the right eye of albino New Zealand white rabbits is measured at pre-administration baseline, as well as post-administration of Alphagan® P as drops, post-administration of a composition comprising brimonidine and a penetration enhancer as a mist, and post-administration of a composition in accordance with the principles of the present invention comprising brimonidine or a salt thereof and being devoid of a penetration enhancer as a mist. Measurement is performed using a laser Doppler retinal blood flow instrument (CLBF 100, Canon Inc., Tokyo, Japan) based on the principle of bidirectional laser Doppler velocimetry as described by Costa V P et al in Prog in Retinal and Eye Res 2003, 22, 769-805 or Yoshida A et al in Am. J. Opthalmol. 2003, 135, 356-361, both which are included by reference as if fully set-forth herein. In this instrument, the measuring laser beam is locked onto the target blood vessel during eye movements through an eye-tracking feedback and control system. Doppler-shifted laser light scattered from a retinal vessel is analyzed to determine centerline blood velocity. The blood column diameter is simultaneously measured, and the blood flow rate at the measurement site is automatically calculated, as described by Yoshida A et al in Am. J. Opthalmol. 2003, 135, 356-361.

Venous blood flow, which is known to be directly correlated to arterial blood flow, is measured, since retinal veins have a larger diameter than retinal arteries, facilitating better locking of the measuring laser beams onto the target vessel. This is especially the case in rabbits, which retinal arteries are very narrow.

The beam from a red 675-nm diode laser is used for velocity measurement, emitted from a fundus camera-like measuring head. The Doppler-shifted light scattered from the flowing blood cells in the target vessel is detected simultaneously in two directions, separated by a fixed angle. The signals from two photomultiplier tube detectors undergo computer-controlled spectrum analysis, and sequential measurements of velocity are performed automatically. Results are acquired at 50 measurements per second for 2 seconds. A tracking stripe provided by a green 543-nm HeNe laser oriented perpendicular to the target vessel is used to measure the diameter of the retinal vessel. Diameter is determined automatically by computer analysis of the signal produced by the image of the vessel on the CCD sensor using the half height of the transmittance profile to define the blood column edge. Diameter measurements are corrected for the axial length of the eye (operator input) and refractive error of the eye, which is measured by the CLBF itself.

The instrument is focussed on the rabbit retina and the blood flow velocity in selected veins of the untreated eye is measured to obtain a baseline reading. After obtaining the baseline reading, a test composition is administered to the anterior surface of the eye. For three rabbits, 10 microliter of the Alphagan® P composition are administered using a standard eyedropper. For three rabbits, 350 microliters of a composition comprising brimonidine and a penetration enhancer are administered as a mist over two minutes. For three rabbits, 350 microliters of a concentration comprising brimonidine and being devoid of a penetration enhancer are administered as a mist over two minutes. Pharmaceutical compositions are ophthalmically administered as a mist using a nebulizer device such as described in PCT publication WO2006/082588 of the Applicant, in U.S. Pat. No. 6,748,977, in US Patent Application 2007/0119968, in Collins J F et al in American Journal of Ophthamology 2007, 144(1), 137-139 or the ophthalmic delivery device by Optimyst, Llc (West Islip, N.Y., USA).

Following administration of the composition, the blood flow velocity in the same veins is measured.

Administration of each of the test compositions results in a substantial increase in retinal blood flow. Rabbits to which the composition is administered as drops show obvious signs of ocular irritation that include eye closure and squealing. Rabbits to which composition is administered as mist do not display overt signs of ocular irritation.

It may therefore be concluded that administration of brimonidine as a mist is as effective as administration in the form of eye-drops, as known in the art, but without the associated discomfort. It may further be concluded that mist compositions which are devoid of a penetration enhancer are as effective as those in which a penetration enhancer is present.

### Example 2

### Effect of Compositions of the Present Invention on Ocular Pressure

A pharmaceutical composition including 0.15% brimonidine tartrate in an ophthalmically acceptable carrier, devoid of a penetration enhancer is applied three times daily as a mist using a nebulizer, as described in Example 1, to a subject suffering from ocular hypertension. Marked reduction of the intraocular pressure is observed.

### Example 3

### Exemplary Compositions

Exemplary pharmaceutical compositions in accordance with the principles of the present invention include brimonidine or a pharmaceutically salt thereof as active ingredient in an ophthalmically-acceptable carrier suitable for administration as a mist.

Specific examples compositions comprising brimonidine tartrate, sodium borate, boric acid, sodium chloride, potassium chloride, calcium chloride, magnesium chloride and sodium perborate in an aqueous solution with HCl and/or NaOH added to adjust the pH to 6.6-8.0.

Embodiments of the invention have been described herein primarily with reference to treatment of living human subjects. It is understood, however, that embodiments of the invention are performed for the veterinary treatment of a non-human mammal, especially horses, cats, dogs, cows, sheep and pigs.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the invention.

Section headings are used herein to ease understanding of the specification and should not be construed as necessarily limiting.

1.-22. (canceled)

23. A method for the treatment of a condition of the eye, comprising: administering an effective amount of a pharmaceutical composition comprising brimonidine or a pharmaceutically acceptable salt thereof in an ophthalmically-acceptable carrier to the eye of a subject as a mist, said composition substantially devoid of a penetration enhancer thereby treating the condition.

24. A method of treatment of a condition of the eye, comprising:
a) providing a pharmaceutical composition comprising brimonidine or a pharmaceutically acceptable salt thereof, and an ophthalmically acceptable carrier, said composition substantially devoid of a penetration enhancer;
b) generating a mist of said composition; and
c) contacting said mist with an anterior surface of the eye of a subject.

25. The method of claim 23, wherein said condition is selected from the group consisting of a condition susceptible to stimulation of retrobulbar blood flow and a condition susceptible to lowering of intraocular blood pressure.

26. The method of claim 25, wherein said condition is susceptible to stimulation of retrobulbar blood flow is selected from the group consisting of: diabetic retinopathy; glaucoma, ocular hypertension, macular degeneration, ocular ischemic syndrome, giant cell arteritis, eye occlusions, central retinal artery occlusion (CRAO), central retinal vein occlusion (CRVA), ischemic optic neuropathy, optic neuritis, neuromyelitis optica and neuroretinitis.

27. The method of claim 25, wherein said condition susceptible to lowering of intraocular blood pressure is selected from the group consisting of: glaucoma and ocular hypertension.

28. The method of claim 27, wherein said glaucoma comprises open angle glaucoma.

29. The method of claim 23, wherein said subject is a human.

30. The method of claim 23, wherein said subject is a non-human animal.

31. The method of claim 23, wherein said mist comprises particles having a mean particle diameter of less than about 20 micrometers.

32. The method of claim 31, wherein said mist comprises particles having a mean particle diameter of less than about 10 micrometers.

33. The method of claim 23, wherein said pharmaceutical composition further comprises a component selected from the group consisting of: buffering agents, pH-adjusting agents, preservatives, and solubilizers.

34. The method of claim 33, wherein said buffering agent is selected from the group consisting of: borate buffers, citrate buffers, acetic acid/sodium acetate buffers, phosphoric acid/sodium phosphate buffers, mannitol, and combinations thereof.

35. The method of claim 33, wherein said pH-adjusting agent is selected from the group consisting of: adipic acid, boric acid, citric acid, glycine, calcium hydroxide, magnesium aluminometasilicates, hydrochloric acid, lactic acid, phosphoric acid, sodium hydroxide, sorbic acid, sulfuric acid and tartaric acid, derivatives thereof, salts thereof, and combinations thereof.

36. The method of claim 33, wherein said preservative is selected from the group consisting of: propylene glycols, sodium propionate, sodium perborate, chlorine dioxide, vitamin E, vitamin E acetate and derivatives, esters, salts, and combinations thereof.

37. The method of claim 33, wherein said solubilizer is selected from the group consisting of: citric acid, ethylenediamine-tetraacetate, sodium meta-phosphate, succinic acid, urea, cyclodextrin, polyvinylpyrrolidone, diethylammonium-ortho-benzoate, micelle-forming solubilizers, TWEEN, SPANS, polyoxyethylene sorbitan fatty acid ester, polyoxyethylene n-alkyl ethers, n-alkyl amine n-oxides, poloxamers, phospholipids and cyclodextrins, and combinations thereof.

38. The method of claim 23, wherein said pharmaceutical composition further comprises a bioadhesive or viscosity modifier.

39. The method of claim 38, wherein said bioadhesive or viscosity modifier is selected from the group consisting of: polyvinyl alcohol, thiolated poly acrylic acid, carbomer and gellan gum, methylcellulose and polyvinylpyrrolidone or combinations thereof.

40. A device for ophthalmic administration of a pharmaceutical composition, comprising:
a) a composition reservoir configured for functional association with a nebulizing device; and
b) a pharmaceutical composition comprising brimonidine or a pharmaceutically acceptable salt thereof and an ophthalmically acceptable carrier contained in said reservoir, wherein said composition is substantially devoid of a penetration enhancer,
wherein said nebulizing device includes a nebulizer configured to nebulize said pharmaceutical composition to generate an ophthalmically administrable mist.

41. A pharmaceutical composition, comprising:
a) brimonidine or a pharmaceutically acceptable salt thereof; and
b) an ophthalmically-acceptable carrier
the composition configured for ophthalmic administration as a mist; and
the composition substantially devoid of a penetration enhancer.
